(19) United States
(12) Patent Application Publication
(10) Pub. No.: US 2010/0016609 A1
(43) Pub. Date: Jan. 21, 2010

(54) METHODS FOR THE PREPARATION OF AZOLE COMPOUNDS

(75) Inventors: Antonina Aristotelevena Nikitenko, Tarrytown, NY (US); Gulnaz Khafizova, West Nyack, NY (US); Jonathan Laird Gross, Cranbury, NJ (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/501,709

(22) Filed: Jul. 13, 2009

Related U.S. Application Data
(60) Provisional application No. 61/080,746, filed on Jul. 15, 2008.

(51) Int. Cl. C07D 413/10

(52) U.S. Cl. 548/236

(57) ABSTRACT

The present invention is directed to processes, compositions and methods associated with the preparation of azole derivatives of formula I:

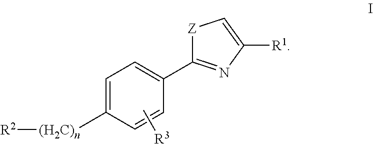

### CROSS-REFERENCE TO RELATED APPLICATIONS

This application claims the benefit under 35 U.S.C. §119(e) to co-pending U.S. provisional application No. 61/080,746 filed Jul. 15, 2008, which is hereby incorporated by reference in its entirety.

### FIELD

The present invention relates to azole compounds, derivatives, methods of their preparation, intermediate compounds, by-products, adducts and mixtures present in the preparation of the azole compounds.

### BACKGROUND

Compounds described in U.S. Application Publication No. 2009-0023707, which is hereby incorporated by reference in its entirety, are potent Histamine-3 (H₃) receptor antagonists that may improve cognitive performance in disease states such as neurodegeneration, cognitive impairment, Alzheimer's disease, Parkinson's disease, dementia, psychosis, depression, attention deficit disorder (ADD)/attention deficit hyperactivity disorder (ADHD), schizophrenia, obesity and sleep disorders.

Despite the exploration of a variety of chemistries to provide therapies based on these H₃ inhibitors, a continuing need exists for preparations which are efficient and amenable to large-scale syntheses. A need also exists for preparations which provide compounds free of impurities and any potentially harmful side-products.

### SUMMARY

The present invention is directed to azole compounds, which are H₃ inhibitors, compositions containing these compounds, processes for their preparation, and intermediate compounds, compositions, by-products, adducts and mixtures present in their preparation.

One particular aspect of the invention provides a process for the preparation of a compound of formula I:

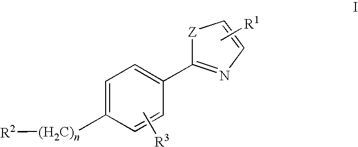

wherein,

R¹ is independently at each occurrence in the process H, halo, hydroxy, —CO₂(C₁-C₃ alkyl), C₁-C₆ alkyl, C₁-C₆ acyl, C₁-C₆ alkoxy, wherein each C₁-C₆ alkyl, C₁-C₆ acyl, C₁-C₆ alkoxy, is substituted with 0-4 substituents independently selected from the group consisting of C₁-C₄ alkyl, C₃-C₁₀ cycloakyl, C₂-C₆ alkenyl, C₂-C₆ alkynyl, halo, nitro, cyano, hydroxy, phenyl, 5-14 membered heterocyclyl or heteroaryl, —N(R^{a})₂, —C(O)R^{b}, —OR^{c} and —S(O)ₚR^{d};

R² is independently at each occurrence in the process a 5-14 membered heteroaryl substituted with 0-4 substituents independently selected from the group consisting of C₁-C₄ alkyl, C₃-C₁₀ cycloakyl, C₂-C₆ alkenyl, C₂-C₆ alkynyl, halo, nitro, cyano, hydroxy, phenyl, a 5-14 membered heterocyclyl or heteroaryl, —N(R^{a})₂, —C(O)R^{b}, —OR^{c} and —S(O)ₚR^{d};

R³ is independently at each occurrence in the process H, halo, nitro, cyano, hydroxy, S(O)ₚR^{d}, —N(R^{a})₂, C₁-C₆ alkyl, C₁-C₆ acyl, C₁-C₆ alkoxy, C₆-C₁₀ aryl, a 5-14 membered heteroaryl or heterocyclyl, or C₃-C₁₀ cycloalkyl, wherein each C₁-C₆ alkyl, C₁-C₆ acyl, C₁-C₆ alkoxy, C₆-C₁₀ aryl, 5-14 membered heteroaryl or heterocyclyl, or C₃-C₁₀ cycloalkyl is substituted with 0-4 substituents independently selected from the group consisting of C₁-C₄ alkyl, C₃-C₁₀ cycloakyl, C₂-C₆ alkenyl, C₂-C₆ alkynyl, halo, nitro, cyano, hydroxy, phenyl, a 5-14 membered heterocyclyl or heteroaryl, —N(R^{a})₂, —C(O)R^{b}, —OR^{c} and —S(O)ₚR^{d};

Z is O or S;

each R^{a} is independently H, C₁-C₄ alkyl optionally substituted with halo, phenyl, —CHO, —C(O)(C₁-C₄ alkyl) or —CO₂(C₁-C₄ alkyl);

each R^{b} is independently H, —OH, —O(C₁-C₄), C₁-C₄ alkyl optionally substituted with halo, phenyl, —NH₂, —NH(C₁-C₄ alkyl) or —N(C₁-C₄ alkyl)₂;

each R^{c} is independently H, C₁-C₄ alkyl, C₁-C₄ haloalkyl, phenyl, —CHO or —C(O)(C₁-C₄ alkyl);

each R^{d} is independently C₁-C₄ alkyl, C₁-C₄ haloalkyl, phenyl or —OH;

each p is independently 0, 1 or 2; and

n is 0 or 1 ; or

a tautomer, stereoisomer or pharmaceutically acceptable salt thereof;

wherein the process comprises reacting a compound of formula IB or tautomer thereof:

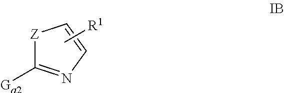

wherein Gₐ₂ is an activating group;

with a compound of formula IA:

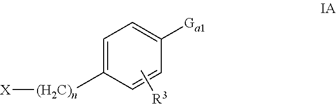

wherein,

Gₐ₁ is an activating group; and

(a) X is R², to form the compound of formula I; or

(b) X is Gₐ₃, thereby forming the compound of IC or tautomer thereof; or X is a hydroxy group and the process further comprises activating the hydroxy group X to form a compound of formula IC or tautomer thereof:

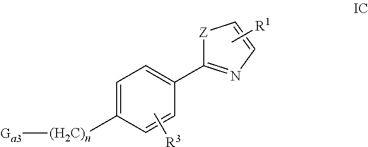

wherein,

Gₐ₃ is an activating group;

(i) optionally activating a compound of the formula H—R² to form activated-R²; and

(ii) reacting H—R² or activated-R² with the compound of formula IC to form the compound of formula I.

Other objects, features and advantages of the present invention will become apparent from the following detailed description. It should be understood, however, that the detailed description and the specific examples, while indicating embodiments of the invention, are given by way of illustration only, since various changes and modifications within the spirit and scope of the invention will become apparent to those skilled in the art from this detailed description.

### DETAILED DESCRIPTION

The following definitions are provided for the full understanding of terms and abbreviations used in this specification.

As used herein and in the appended claims, the singular forms “a,” “an,” and “the” include the plural reference unless the context clearly indicates otherwise. Thus, for example, “a compound” is a reference to one or more compounds and equivalents thereof known to those skilled in the art, “a catalyst” refers to one or more catalysts and equivalents thereof known to those skilled in the art, and so forth.

The abbreviations in the specification correspond to units of measure, techniques, properties, or compounds as follows: “min” means minutes, “h” means hour(s), “μL” means microliter(s), “mL” means milliliter(s), “mM” means millimolar, “M” means molar, and “mmole” means millimole(s).

The terms “component,” “composition,” “composition of compounds,” “compound,” “drug,” or “pharmacologically active agent” or “active agent” or “medicament” are used interchangeably herein to refer to a compound or compounds or composition of matter which, when administered to a subject (human or animal) induces a desired pharmacological and/or physiologic effect by local and/or systemic action.

Within the present invention, the compounds may be prepared in the form of salts and pharmaceutically acceptable salts. As used herein, the term “pharmaceutically acceptable salts” refers to salts prepared from pharmaceutically acceptable non-toxic acids, including inorganic salts and organic salts. Suitable non-organic salts include inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferred is the hydrochloride salt. In the preparation of intermediates, any compatible salt can be used, toxic or non-toxic, for example Bu₄N+ salts.

“Administering,” as used herein, means either directly administering a compound or composition of the present invention, or administering a prodrug, derivative or analog which will form an equivalent amount of the active compound or substance within the body.

The term “subject” or “patient” refers to an animal including the human species that is treatable with the compounds, compositions, and/or methods of the present invention.

Unless indicated otherwise, the nomenclature of substituents that are not explicitly defined herein are arrived at by naming the terminal portion of the functionality followed by the adjacent functionality toward the point of attachment. For example, the substituent “arylalkoxycabonyl” refers to the group (aryl)-(alkyl)-O—C(O)—.

It is understood that the above definitions are not intended to include impermissible substitution patterns (e.g., methyl substituted with 5 fluoro groups). Such impermissible substitution patterns are well known to the skilled artisan.

At various places in the present specification, substituents of compounds are disclosed in groups or in ranges. It is specifically intended that the description include each and every individual subcombination of the members of such groups and ranges. For example, the term “C₁-₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of another example, the term “5-14 membered heteroaryl group” is specifically intended to individually disclose a mono- or polycyclic heteroaryl group having 5, 6, 7, 8, 9, 10, 11, 12, 13, 14, 5-14, 5-13, 5-12, 5-11, 5-10, 5-9, 5-8, 5-7, 5-6, 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, and 13-14 ring atoms.

The term “protecting group” or “Gₚ” with respect to amine groups, hydroxyl groups and sulfhydryl groups refers to forms of these functionalities which are protected from undesirable reaction with a protecting group known to those skilled in the art, such as those set forth in Protective Groups in Organic Synthesis, Greene, T. W.; Wuts, P. G. M., John Wiley & Sons, New York, N.Y., (3rd Edition, 1999), the entire disclosure of which is herein incorporated by reference, which protecting groups can be added or removed using the procedures set forth therein. Examples of protected hydroxyl groups include, but are not limited to, silyl ethers such as those obtained by reaction of a hydroxyl group with a reagent such as, but not limited to, t-butyldimethyl-chlorosilane, trimethylchlorosilane, triisopropylchlorosilane, triethylchlorosilane; substituted methyl and ethyl ethers such as, but not limited to methoxymethyl ether, methythiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoracetate. Examples of protected amine groups include, but are not limited to, amides such as, formamide, acetamide, trifluoroacetamide, and benzamide; carbamates; e.g. BOC; imides, such as phthalimide, Fmoc, Cbz, PMB, benzyl, and dithiosuccinimide; and others. Examples of protected or capped sulfhydryl groups include, but are not limited to, thioethers such as S-benzyl thioether, and S-4-picolyl thioether; substituted S-methyl derivatives such as hemithio, dithio and aminothio acetals; and others.

“As used herein, an “an activating group” is a group that, when bound to a center, increases the reactivity at that center. Non-limiting examples of an activating group include a substituent bound to an electrophilic center and capable of being displaced by a nucleophile; a substituent bound to a nucleophilic center and capable of being displaced by an electrophile; a substituent capable of being displaced by a radical; or a substituent bound to a center wherein, following gain or loss of an eletron, the substituent is capable of leaving as an anion or cation with formation of a radical at the center.

As used herein, “activating” a compound refers to reacting the compound at a center with a reagent to introduce at the center an activating group, wherein the activating group is optionally converted to another activating group in one or more steps. Examples of activating include halogenation at a carbon center, optionally followed by hydroboration wherein the halogen group is converted to an optionally sustituted borane; tosylation, mesylation, or triflation at an oxygen center; and nitration at a carbon center optionally followed by reduction of the nitro group to an amino group and conversion of the amino group to a diazo group.

The term “deprotecting” refers to removal of a protecting group, such as removal of a benzyl or BOC group bound to an amine. Deprotecting may be preformed by heating and/or addition of reagents capable of removing protecting groups. In preferred embodiments, the deprotecting step involves addition of an acid, base, reducing agent, oxidizing agent, heat, or any combination thereof. One preferred method of removing BOC groups from amino groups is to add HCl in ethyl acetate. Many deprotecting reactions are well known in the art and are described in Protective Groups in Organic Synthesis, Greene, T. W., John Wiley & Sons, New York, N.Y., (3^{rd} Edition, 1999), the entire disclosure of which is herein incorporated by reference.

A “Suzuki coupling”, refers to a palladium-catalysed cross coupling reaction between organoboronic acids/esters and activating groups, preferably halides.

“Alkyl” refers to monovalent saturated aliphatic hydrocarbyl groups having from 1 to 10 carbon atoms and preferably 1 to 8 carbon atoms (C₁-C₈ alkyl) and more preferably, 1 to 6 carbon atoms (C₁-C₆ alkyl). This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkoxy” refers to the group —O-alkyl wherein alkyl is defined herein. Preferred alkoxy groups have 1 to 6 carbon atoms (C₁-C₆ alkoxy). Alkoxy includes, by way of example, methoxy, ethoxy, n-propoxy, isopropoxy, n-butoxy, t-butoxy, sec-butoxy, and n-pentoxy.

“Amino” refers to the group —NH₂.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups are C₆-C₁₀ aryl groups and include phenyl and naphthyl.

“Alkenyl” refers to alkenyl groups having from 2 to 6 carbon atoms (C₂-C₆ alkenyl) and preferably 2 to 4 carbon atoms (C₂-C₄ alkenyl) and having at least 1 and preferably from 1 to 2 sites of alkenyl unsaturation. Such groups are exemplified, for example, by vinyl, allyl, and but-3-en-1-yl.

“Alkynyl” refers to alkynyl groups having from 2 to 6 carbon atoms (C₂-C₆ alkynyl) and preferably 2 to 3 carbon atoms (C₂-C₃ alkynyl) and having at least 1 and preferably from 1 to 2 sites of alkynyl unsaturation.

“Acyl” refers to the groups H—C(O)—, alkyl-C(O)—, alkenyl-C(O)—, alkynyl-C(O)—, cycloalkyl-C(O)—, cycloalkenyl-C(O)—, aryl-C(O)—, 5-14 membered heteroaryl-C(O)—, 5-14 membered heterocyclic-C(O)—, wherein alkyl, alkenyl, alkynyl, cycloalkyl, cycloalkenyl, aryl, heteroaryl, and heterocyclic are as defined herein. Acyl includes the “acetyl” group CH₃C(O)—.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Preferred cycloalkyl groups have 3 to 6 carbon atoms (C₃-C₆ cycloalkyl). Examples of suitable cycloalkyl groups include, for instance, adamantyl, cyclopropyl, cyclobutyl, cyclopentyl, cyclohexyl, and cyclooctyl.

“Cycloalkenyl” refers to cyclic alkyl groups of from 3 to 10 carbon atoms having single or multiple cyclic rings including fused, bridged, and spiro ring systems which contain at least one double bond. Preferred cycloalkenyl groups have 3 to 6 carbon atoms (C₃-C₆ cycloalkenyl) and contain one double bond. Examples of suitable cycloalkenyl groups include, for instance, cyclobutenyl, cyclopentenyl, cyclohexenyl, and cyclooctenyl.

“Halo” or “halogen” refers to fluoro, chloro, bromo and iodo.

“Hydroxy” or “hydroxyl” refers to the group —OH.

“Heteroaryl” refers to a monocyclic or fused polycyclic aromatic group with 1 to 4 heteroatoms selected from the group consisting of oxygen, nitrogen and sulfur within the ring, wherein the nitrogen of the heteroaryl group is optionally oxidized to an N-oxide (N→O) moiety. Preferably, the heteroaryl is a 5 to 14 membered heteroaryl.

Preferred heteroaryls include pyridinyl, pyrrolyl, thiophenyl, furanyl, benzamidazolyl, indolyl, quinazolinyl and quinolinyl.

“Heterocyclyl” refers to a saturated or a non-aromatic, unsaturated group having one or more (fused if more than one) rings with from 1 to 4 hetero atoms selected from the group consisting of nitrogen, sulfur or oxygen within the ring, wherein the nitrogen and/or sulfur atom(s) of the heterocyclic group are optionally oxidized to N-oxide, sulfinyl, and/or sulfonyl moieties. Preferably, the heteroaryl is a 3 to 14 membered heterocyclyl. Examples of heterocycle groups include, but are not limited to, piperazine, morpholinyl, thiomorpholinyl (also referred to as thiamorpholinyl), 1,1-dioxothiomorpholinyl, piperidine, pyrrolidine, tetrahydrofuranyl, quinuclidinyl, octahydropyrrolo[1,2-a]pyrazine, 4,7-diazaspiro[2.5]octane, and tetrahydroquinolinyl.

A “fused aromatic ring” as used herein is an aromatic ring formed by taking together two substituents of a second ring, each substituent being bonded to a carbon atom of the second ring, with the two carbon atoms of the second ring, wherein the two carbon atoms of the second ring are connected by a direct bond. An example of a fused aromatic ring is a fused benzene ring, which may be optionally substituted as disclosed herein.

A “fused heteroaromatic ring” as used herein is a heteroaromatic ring formed by taking together two substituents of a second ring, each substituent being bonded to a carbon atom of the second ring, with the two carbon atoms of the second ring, wherein the two carbon atoms of the second ring are connected by a direct bond, and wherein the fused ring contains 1 to 4 heteroatoms selected from the group consisting of oxygen, nitrogen and sulfur within the ring. An example of a fused aromatic ring is a fused pyridine ring, which may be optionally substituted as disclosed herein.

“Nitro” refers to the group —NO₂.

“Oxo” refers to the atom (═O) or (—O—). As an activating group, ‘oxo’ groups are amenable to reductive amination by nucleophilic amine groups to form alkylamino or aminoalkyl substituents. Preferably, the reductive amination step takes place in the presence of a boron-containing reducing agent.

“Stereoisomer” or “stereoisomers” refer to compounds that differ in the chirality or atomic connectivity at one or more stereocenters. Stereoisomers include enantiomers, diastereomers as well as cis-trans (E/Z) isomerism.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Patient” or “subject” refers to mammals and includes humans and non-human mammals, such as dogs, cats, mice, rats, cows, rabbits and monkeys.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, ascorbate, phosphate, acetate, maleate, and oxalate.

The pharmaceutically acceptable salts are prepared by contacting a compound, such as the compound of formula (I) with an acid or ion pair such as selected from hydrochloric acid, hydrobromic acid, acetic acid, phosphoric acid, boric acid, perchloric acid, tartaric acid, maleic acid, citric acid, methanesulfonic acid, ascorbic acid and the like. A solvent employed may be selected from ketones such as acetone, diethyl ketone, methyl ethyl ketone or their mixtures, methanol, ethanol, n-hexane, ethylacetate, benzene, diethylamine, formaldehyde, chloroform, dichloromethane or mixture thereof.

“Treating” or “treatment” of a disease in a subject refers to inhibiting the disease, arresting its development; or ameliorating or causing regression of the disease.

“Modulating 5-HT₆ receptor activity” refers to affecting (i.e. inhibition or stimulation) processes or signaling events associated with the 5-HT₆ receptor. Specifically, inhibition of 5-HT₆ increases levels of acetylcholine and glutamate in the brain, whereas 5-HT₆ receptor agonism or stimulation results in increased cellular cAMP.

A “CNS disease” or “CNS disorder” a disease or disorder affecting or originating in the central nervous system, preferably a disease related to 5-HT₆ activity or affected by 5-HT₆ modulation. Particular CNS diseases or disorder include psychoses, anxiety, depression, epilepsy, migraine, cognitive disorders, sleep disorders, feeding disorders, anorexia, bulimia, binge eating disorders, panic attacks, disorders resulting from withdrawal from drug abuse, schizophrenia, gastrointestinal disorders, irritable bowel syndrome, memory disorders, obsessive compulsive disorders, Alzheimer's disease, Parkinson's disease, Huntington's chorea, schizophrenia, attention deficit hyperactive disorder, ADD, ADHD, Restless Legs Syndrome, MCI, stroke, neurodegenerative diseases characterized by impaired neuronal growth, and pain.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

One aspect of the present invention provides a process for the preparation of a compound of formula I:

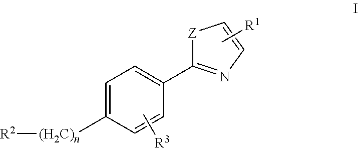

wherein,

R¹ is independently at each occurrence in the process H, halo, hydroxy, —CO₂(C₁-C₃ alkyl), C₁-C₆ alkyl, C₁-C₆ acyl, C₁-C₆ alkoxy, wherein each C₁-C₆ alkyl, C₁-C₆ acyl, C₁-C₆ alkoxy, is substituted with 0-4 substituents independently selected from the group consisting of C₁-C₄ alkyl, C₃-C₁₀ cycloakyl, C₂-C₆ alkenyl, C₂-C₆ alkynyl, halo, nitro, cyano, hydroxy, phenyl, 5-14 membered heterocyclyl or heteroaryl, —N(R^{a})₂, —C(O)R^{b}, —OR^{c} and —S(O)ₚR^{d};

R² is independently at each occurrence in the process a 5-14 membered heteroaryl substituted with 0-4 substituents independently selected from the group consisting of C₁-C₄ alkyl, C₃-C₁₀ cycloakyl, C₂-C₆ alkenyl, C₂-C₆ alkynyl, halo, nitro, cyano, hydroxy, phenyl, a 5-14 membered heterocyclyl or heteroaryl, —N(R^{a})₂, —C(O)R^{b}, —OR^{c} and —S(O)ₚR^{d};

R³ is independently at each occurrence in the process H, halo, nitro, cyano, hydroxy, S(O)ₚR^{d}, —N(R^{a})₂, C₁-C₆ alkyl, C₁-C₆ acyl, C₁-C₆ alkoxy, C₆-C₁₀ aryl, a 5-14 membered heteroaryl or heterocyclyl, or C₃-C₁₀ cycloalkyl, wherein each C₁-C₆ alkyl, C₁-C₆ acyl, C₁-C₆ alkoxy, C₆-C₁₀ aryl, 5-14 membered heteroaryl or heterocyclyl, or C₃-C₁₀ cycloalkyl is substituted with 0-4 substituents independently selected from the group consisting of C₁-C₄ alkyl, C₃-C₁₀ cycloakyl, C₂-C₆ alkenyl, C₂-C₆ alkynyl, halo, nitro, cyano, hydroxy, phenyl, a 5-14 membered heterocyclyl or heteroaryl, —N(R^{a})₂, —C(O)R^{b}, —OR^{c} and —S(O)ₚR^{d};

Z is O or S;

each R^{a} is independently H, C₁-C₄ alkyl optionally substituted with halo, phenyl, —CHO, —C(O)(C₁-C₄ alkyl) or —CO₂(C₁-C₄ alkyl);

each R^{b} is independently H, —OH, —O(C₁-C₄), C₁-C₄ alkyl optionally substituted with halo, phenyl, —NH₂, —NH(C₁—C₄ alkyl) or —N(C₁-C₄ alkyl)₂;

each R^{c} is independently H, C₁-C₄ alkyl, C₁-C₄ haloalkyl, phenyl, —CHO or —C(O)(C₁-C₄ alkyl);

each R^{d} is independently C₁-C₄ alkyl, C₁-C₄ haloalkyl, phenyl or —OH;

each p is independently 0, 1 or 2; and

n is 0 or 1 ; or

a tautomer, stereoisomer or pharmaceutically acceptable salt thereof;

wherein the process comprises reacting a compound of formula IB or tautomer thereof:

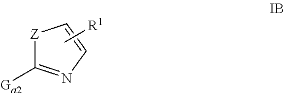

wherein Gₐ₂ is an activating group;

with a compound of formula IA:

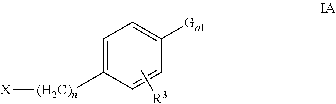

wherein,

Gₐ₁ is an activating group; and

(a) X is R², to form the compound of formula I; or

(b) X is Gₐ₃, thereby forming the compound of IC or tautomer thereof; or X is a hydroxy group and the process further comprises activating the hydroxy group X to form a compound of formula IC or tautomer thereof:

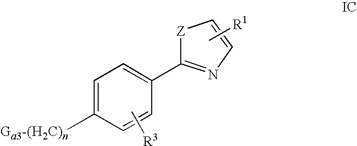

wherein,

Gₐ₃ is an activating group;

(i) optionally activating a compound of the formula H—R² to form activated-R²; and

(ii) reacting H—R² or activated-R² with the compound of formula IC to form the compound of formula I.

In a more particular embodiment, R¹ is —CO₂(C₁-C₃ alkyl). More particularly, R¹ is —CO₂CH₂CH₃. In another more particular embodiment, the process further comprises:
reducing the —CO₂(C₁-C₃ alkyl) group in the compound of formula IC to form a reduced-R¹ group.

In another embodiment, the reducing step comprises contacting the —CO₂(C₁-C₃ alkyl) group with a reducing metal hydride; and

wherein the reduced-R¹ group is —CH₂OH

In another embodiment, the reducing metal hydride is lithium aluminum hydride (LiAIH₄).

In another embodiment, Z is O. In another embodiment, R³ is H. In another embodiment, n is 1. In another embodiment, R² is benzimidazolyl substituted with 0-4 substituents independently selected from the group consisting of C₁-C₄ alkyl, C₃-C₁₀ cycloakyl, C₂-C₆ alkenyl, C₂-C₆ alkynyl, halo, nitro, cyano, hydroxy, phenyl, a 5-14 membered heterocyclyl or heteroaryl, —N(R^{a})₂, —C(O)R^{b}, —OR^{c} and —S(O)ₚR^{d}. More particularly, R² is unsubstituted benzimidazol-1-yl.

In another embodiment, each activating group is independently selected from the group consisting of halo, —B(OH)₂, tosylate, mesylate, and triflate. In another embodiment, the activating group is oxo (═O).

In another embodiment, Gₐ₁ is —B(OH)₂. In another embodiment, Gₐ₂ is chloro or bromo. In another embodiment, Gₐ₃ is chloro or bromo.

In another embodiment, the step of reacting a compound of formula IA with a compound of formula IB comprises a Suzuki coupling in the presence of a palladium catalyst. More particularly, the palladium catalyst is not tetrakis(triphenylphosphine)palladium (0). More particular still, the Suzuki coupling is performed in the presence of tris(dibenzylideneacetone) dipalladium (0) and tri(tertbutylphosphonium)tetrafluoroborate. In another embodiment, the Suzuki coupling is performed in a solvent comprising aqueous dioxane and postassium carbonate.

In another embodiment, X is a hydroxy group. In a more particular embodiment thereof, the step of activating the hydroxy group comprises contacting the hydroxyl group with a halogenating agent. More particularly, the halogenating agent is thionyl chloride.

In another embodiment, the step (b)(ii) comprises reacting H—R² with the compound of formula IC in the presence of a base. More particularly, H—R² is 1H-benzo[d]imidazole and the base is sodium hydride (NaH).

In a more particular embodiment of the compound of formula IA, X is R²; and the process further comprises preparing a compound of formula IA by reacting H—R² or activated-R² with a compound of formula ID:

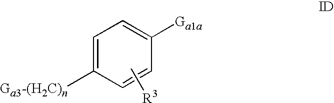

wherein,

Gₐ₃ is an activating group; and

Gₐ₁ₐ is the same activating group as Gₐ₁ in the compound of formula IA, thereby forming the compound of formula IA; or

Gₐ₁ₐ is a different activating group from Gₐ₁ in the compound of formula IA and the process further comprises converting Gₐ₁ₐ to Gₐ₁, thereby forming the compound of formula IA.

In a more particular embodiment, Gₐ₁ₐ is bromo and Gₐ₁ in the compound of formula IA is —B(OH)₂. In another embodiment, the converting step comprises reacting the compound of formula ID with a in the presence of tert-butyllithium and triisopropyl boronic acid (B(OiPr)₃). In another embodiment, Gₐ₃ is bromo.

In another embodiment, R¹ is bound alpha to the N-position, indicating that the compounds of formula I, IB, IC and II have the following structures (respectively):

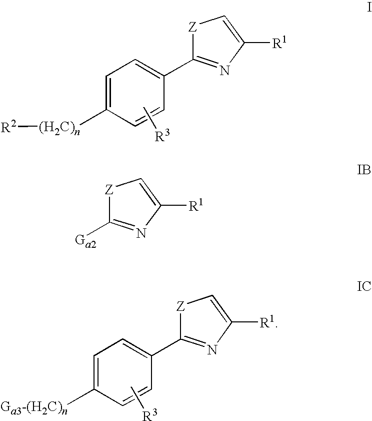

In another embodiment, any of the process steps is performed in a solvent which is independently a protic solvent, an aprotic solvent, a polar solvent, a nonpolar solvent, a protic polar solvent, an aprotic nonpolar solvent, or an aprotic polar solvent. In another embodiment, any of the process steps includes a purification step comprising at least one of: filtration, extraction, chromatography, trituration, or recrystalization. In another embodiment, any of the process steps comprises an analytical step comprising liquid chromatography (LC), mass spectroscopy (MS), liquid chromatography/mass spectroscopy (LC/MS), gas chromatography (GC), gas chromatography/mass spectroscopy (GC/MS), nuclear magnetic resonance (NMR), thin layer chromatography (TLC), melting point (MP) analysis, optical rotation (OR) or elemental analysis.

Another aspect of the invention provides a compound of formula I, IA, IB, IC, ID or II as described herein.

Another aspect of the invention provides a composition comprising:

one or more of the compounds of formula I, IA, IB, IC or ID; and optionally further comprising one or more of: a base, an acid, a solvent, a hydrogenating agent, a reducing agent, an oxidizing agent, or a catalyst.

Some of the compounds of the present invention may contain chiral centers and such compounds may exist in the form of stereoisomers (i.e. enantiomers or diastereomers). The present invention includes all such stereoisomers and any mixtures thereof including racemic mixtures. Racemic mixtures of the stereoisomers as well as the substantially pure stereoisomers are within the scope of the invention. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron, 33:2725 (1977); Eliel, E. L. Stereochemistry of Carbon Compounds, (McGraw-Hill, NY, 1962); Wilen, S. H. Tables of Resolving Agents and Optical Resolutions, p. 268 (E. L. Eliel, Ed., University of Notre Dame Press, Notre Dame, IN 1972), the entire disclosures of which are herein incorporated by reference.

The term “substantially pure,” as used herein, refers to at least about 90 mole %, more preferably at least about 95 mole %, and most preferably at least about 98 mole % of the desired product.

Further, the compounds of formula I may exist in unsolvated as well as in solvated forms with pharmaceutically acceptable solvents such as water, ethanol, and the like. In general, the solvated forms are considered equivalent to the unsolvated forms for the purpose of the present invention.

The compounds of formula I can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

Unless indicated otherwise, a particular R-group present on a compound in a synthetic process can be any substituent defined for that R-group and is not necessarily identical to the same R-group in a subsequent product or precursor molecule.

Compounds of the present invention are suitably prepared in accordance with the following general description and specific examples. Reagents used in the preparation of the compounds of this invention can be either commercially obtained or can be prepared by standard procedures described in the literature. In accordance with this invention, compounds of formula I may be produced by the following reaction schemes.

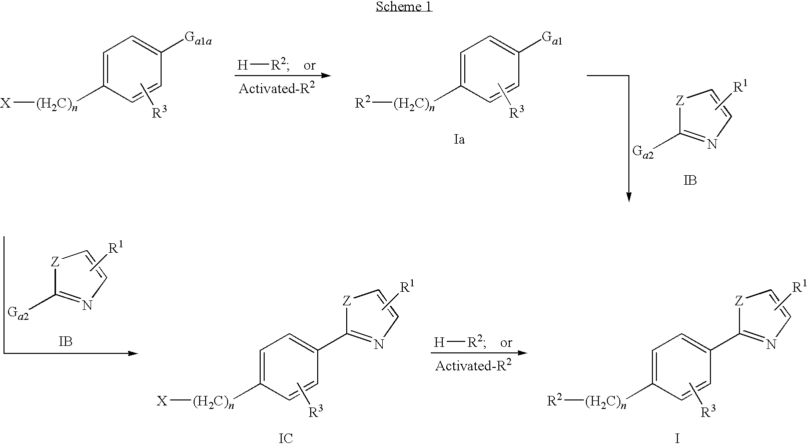

Scheme I depicts the synthesis of the compound of formula I through alternate efficient syntheses. In the synthesis, X is Gₐ₃ in the reaction to form Ia and X is either —OH or Gₐ₃ in the reaction to form IC, wherein Gₐ₁, Gₐ₂ and Gₐ₃ are activating groups as defined herein. Where X is —OH, the reaction optionally includes an additional step after formation of IC, wherein X is activated to form Gₐ₃. In both routes, reaction with IB occurs through a Suzuki coupling. The reaction with H—R² or Activated-R² will be apparent to one skilled in the art. Particularly, wherein R² is connected to through nucleophile (e.g. an amino or oxy group), H—R² can be used. Otherwise R² is activated to effect conjugation.

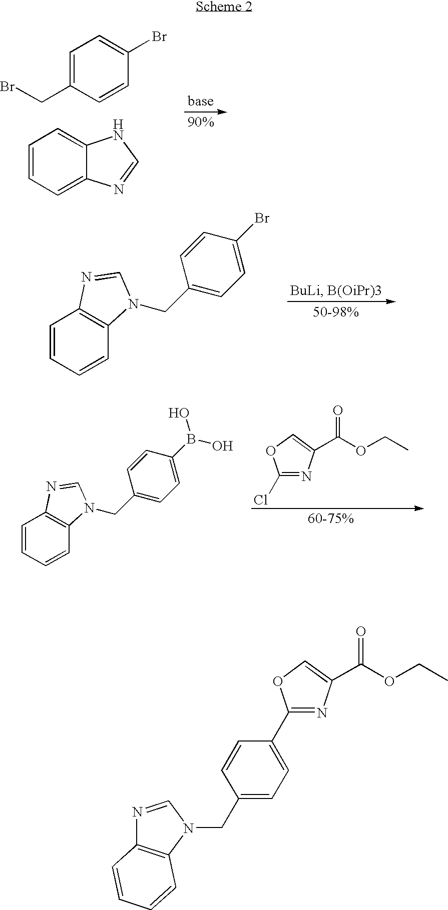

Scheme 2 depicts the synthesis of chlorooxazole ester as a convergent fragment and coupling it with the benzyl-benzimidazole part via Suzuki coupling. In the synthesis, ethyl 2-chloro-1,3-oxazole-4-carboxylate is commercially available from Synthonix Corporation (2713 Connector Drive, Wake Forest, N.C., 27587). In the first step, reaction of benzimidazole with bromobenzyl bromide in ethanol with KOH as a base, provided the target compound, in 90% yield after isolation. The next step, boronic acid formation, was done with tert-butyllithium and triisopropyl borate in THF at −70° C. HPLC indicated complete conversion of the bromide to the target boronic acid; however, its isolation was difficult, as the boronic acid was soluble in both acidic and basic aqueous media. By neutralizing the aqueous solution to pH 7, the desired boronic acid was isolated in 75% yield. However, variation in conditions of isolation resulted in a different form of boronic acid, which behaved differently in the subsequent Suzuki coupling.

Using tetrakis(triphenylphosphine)palladium(0) in Suzuki coupling, dimerization of the boronic acid and hydrolysis of the chlorooxazole ester were observed. Alternatively, performing the reaction with the catalyst, formed in situ from tris(dibenzylideneacetone) dipalladium (0) and tri(tert-butylphosphonium)tetrafluoroborate in dioxane/aqueous potassium carbonate gave the desired product in 75% yield; the side reactions were deboronation of the boronic acid and hydrolysis of the target compound. Results of the reaction considerably varied for different batches of the boronic acid.

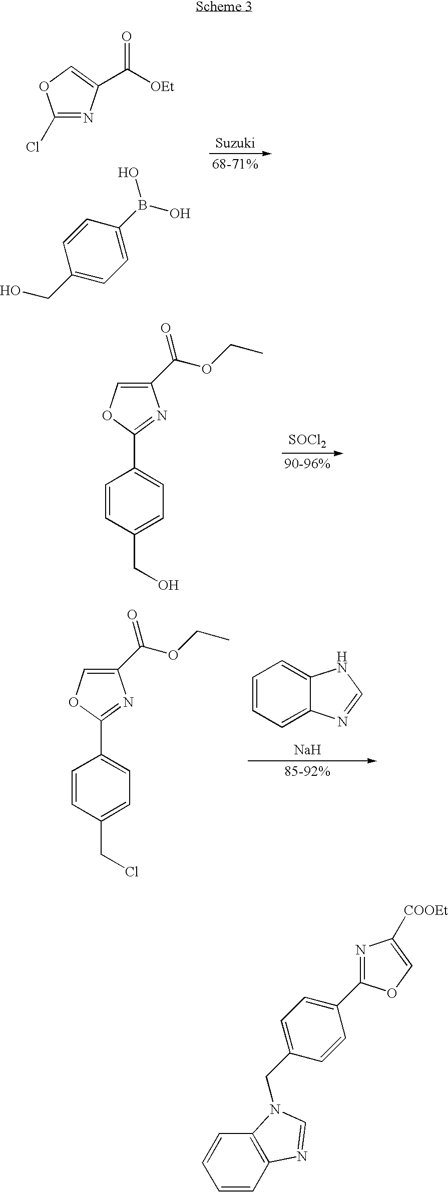

Scheme 3 depicts an alternate synthesis which eliminates the need for isolation/purification of the zwitterionic boronic acid in Scheme 1. In the first step, Suzuki coupling of ethyl 2-chloro-1,3-oxazole-4-carboxylate and 4-(hydroxymethyl)phenylboronic acid using a catalyst formed in situ from tris(dibenzylideneacetone) dipalladium(0) and tri(tert-butylphosphonium)tetrafluoroborate in dioxane/aqueous potassium carbonate gave 71 % yield of the target compound. The hydroxymethyl group was converted to chloromethyl with thionyl chloride with a yield of about 90%. Condensation with benzimidazole was done in DMF, using sodium hydride as a base, and produced the ester in an 86% yield.

### EXAMPLES

### Example 1

### Step 1: Preparation of 1-(4-Bromobenzyl)-1H-benzimidazole

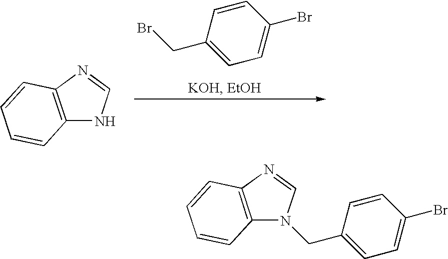

To a solution of benzimidazole (2.4 g, 20.2 mmol) in ethanol (30 mL) was added a solution of KOH (1.18 g, 20 mmol) in 30 ml of ethanol (30 mL) and the reaction mixture was allowed to stir at room temperature overnight. The resulting suspension was diluted with water (50 ml) and the resulting precipitate filtered, washed with water, dried in the vacuum oven at 45° C. overnight to give 5.16 g, 89.8%, of the product, m. p. 93-95° C. ¹H NMR (300 MHz, DMSO-D6, δ): 8.42 (s, 1H), 7.67 (m,1H), 7.54 (d, 2H, J=7.6 Hz), 7.50 (m, 1H), 7.26 (d, 2H, J=7.6 Hz), 7.20(m, 2H), 5.49 (s, 2H).

Step 2: Preparation of [4-(1H-Benzimidazol-1-ylmethyl)phenyl]boronic acid

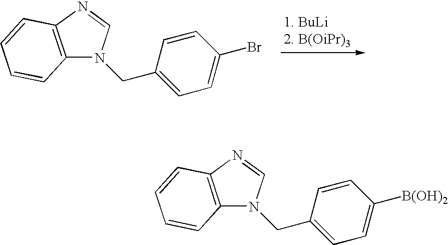

To a solution of 1-(4-bromobenzyl)-1H-benzimidazole (4.3 g, 15 mmol) and triisopropylborate (6.2 g, 7.6 ml, 33 mmol) in anhydrous THF (50 mL) cooled to −70° C. under an inert atmosphere was slowly added lithium tert-butoxide (1.6M in pentane, 28 ml, 45 mmol) and the reaction mixture was stirred at −70° C. for 1 h, then warmed up slowly to −10−0° C., poured into 1N aq. HCl (100 ml), and allowed to stir overnight under nitrogen. The resulting suspension was neutralized to pH 7 with 5.5 N aq. LiOH. The precipitated boronic acid was filtered, washed with minimal amount of cold water, and dried to give 3.71 g (98%) of the target boronic acid. ¹H NMR (300 MHz, DMSO-D6, δ): 8.42 (s, 1H), 8.02 (s, 2H), 7.73 (d, 2H, J=7.8 Hz), 7.66 (m, 1H), 7.49 (m, 1H), 7.25 (d, 2H, J=7.8 Hz), 7.19 (m, 2H), 5.50 (s, 2H).

Step 3: Preparation of Ethyl 2-[4-(1H-benzimidazol-1-ylmethyl)phenyl]-1,3-oxazole-4-carboxylate

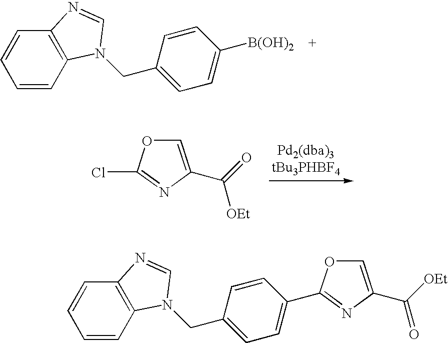

To a suspension of tris(dibenzylideneacetone)dipalladium (0) (230 mg, 0.25 mmol) and tri-tert-butylphosphonium tetrafluoroborate (150 mg, 0.5 mmol) in dioxane (50 ml) and 1N aqueous potassium carbonate (10 ml) degassed with nitrogen was added chlorooxazole carboxylate (1.75. g, 10 mmol) and the reaction mixture allowed to stir for 10 min. A solution of [4-(1H-benzimidazol-1-ylmethyl)phenyl]boronic acid (2.8 g, 11 mmol) in 1N aqueous potassium carbonate (7 mL) was added and the reaction mixture was heated to 86° C. (reflux) for 2 h. The reaction mixture was cooled to room temperature, filtered, diluted with ethyl acetate (50 mL) and washed with conc.aq. ammonium chloride (30 ml). The combined aqueous layers were washed with ethyl acetate and the combined organic layers washed with aqueous sodium bicarbonate, brine, dried over sodium sulphate, filtered, evaporated, and the crude product crystallized from MTBE to give 2.6 g (75%) of the target compound. m.p. 99-101° C.; ¹H NMR (300 MHz, DMSO-D6, δ): 8.92 (s, 1H), 8.45 (d, 2H, J=8.5 Hz), 7.99 (d, 2H, J=8.5 Hz), 7.68 (m, 1H), 7.51 (m, 1H), 7.46 (d, 2H, J=8.5 Hz), 7.21 (m, 2H), 5.61 (s, 2H), 4.31(m, 2H, J=7.2 Hz), 3.32 (s, 2H), 130 (t, 3H, J=7.2 Hz).

1-{4-[4-(pyrrolidin-1-ylmethyl)-1,3-oxazol-2-yl]benzyl}-1H-benzimidazole is prepared from ethyl 2-[4-(1H-benzimidazol-1-ylmethyl)phenyl]-1,3-oxazole-4-carboxylate as described in US Application Pub. No. 2009-0023707, particularly Examples 4-6.

### Example 2

Step 1: Preparation of ethyl 2-[4-(hydroxymethyl)phenyl]-1,3-oxazole-4-carboxylate

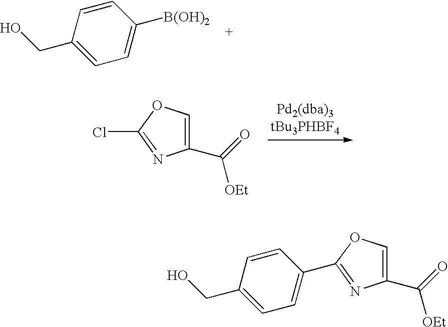

To a suspension of tris(dibenzylideneacetone)dipalladium (0) (3.1 g, 3.36 mmol) and tri-tert-butylphosphonium tetrafluoroborate (1.95 g, 6.7 mmol) in a mixture of dioxane (500 ml) and 1N aqueous potassium carbonate (150 ml) degassed with nitrogen was added chlorooxazole carboxylate (23.5. g, 135 mmol) followed by a suspension of 4-hydroxymethylphenylboronic acid (21.3 g, 135 mmol) in a mixture of dioxane (200 ml, +50 ml wash) and 1N aqueous potassium carbonate (30 ml) continuing to degas with nitrogen and the resulting reaction mixture was stirred at 86° C. (reflux) until determined complete by HPLC (about 2 h). The reaction mixture was allowed to cool to room temperature and filtered through celite, the organic layer separated, and the aqueous layer extracted with ethyl acetate. The dioxane layer was concentrated (avoiding total evaporation to dryness) and combined with the ethyl acetate extract and washed with aq. sodium bicarbonate, brine, dried over sodium sulphate, filtered, evaporated, and triturated with MTBE. The isolated yield of the target compound 22.7 g (68%). m.p. 99-101° C.; ¹H NMR (300 MHz, CDCl3, δ): 8.27 (s, 1H), 8.10 (d, 2H, J=8.2 Hz), 7.47 (d, 2H, J=8.2 Hz), 4.77 (s, 2H), 4.43 (m, 2H, J=7.4 Hz), 1.41 (t, 3H, J=7.4 Hz).

Step 2: Preparation of ethyl 2-[4-(chloromethyl)phenyl]-1,3-oxazole-4-carboxylate

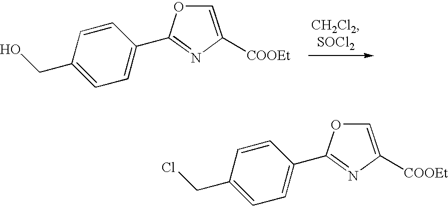

To a mixture of thionyl chloride (15 ml) in methylene chloride (50 ml) at −10° C. was added portionwise ethyl 2-[4-(hydroxymethyl)phenyl]-1,3-oxazole-4-carboxylate (13.6 g, 55 mmol) and the resulting mixture was stirred at room temperature for 3 h. the solvent was removed and the residue was azeotroped with toluene, dissolved in methylene chloride, washed with aqueous sodium bicarbonate, dried over magnesium sulfate, filtered and evaporated to give 14.2 g (96%) of the product as white crystals; m.p. 115-117° C. ¹H NMR (300 MHz, CDCl3, δ): 8.28 (s, 1H), 8.12 (d, 2H, J=8.5 Hz), 7.50 (d, 2H, J=8.5 Hz), 4.62 (s, 2H), 4.43 (m, 2H, J=7.4 Hz), 1.41 (t, 3H, J=7.4 Hz).

Step 3: Preparation of ethyl 2-[4-(1H-benzimidazol-1-ylmethyl)phenyl]-1,3-oxazole-4-carboxylate

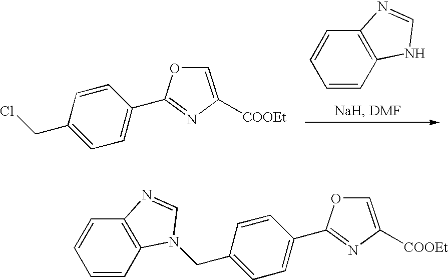

To a solution of benzimidazole (13.5 g, 115 mmol) in DMF (150 ml) was added portionwise sodium hydride (60% suspension in mineral oil, 4.6 g, 115 mmol) and the reaction mixture was allowed to stir for 45 min at room temperature. The reaction mixture was then added to a solution of ethyl 2-[4-(chloromethyl)phenyl]-1,3-oxazole-4-carboxylate (20.3 g, 76.4 mmol) in DMF (150 ml) and the reaction mixture was allowed to stir at room temperature for 2 h. The reaction mixture was poured into ice/water (about 800 ml), stirred for 30 min, and the resulting precipitate filtered through a coarse filter without vacuum suction (if the filtration is bad, it is possible to decant the aqueous/DMF layer from the organic precipitate), the precipitate dissolved in methylene chloride, washed with aq. ammonium chloride, water, dried over sodium sulphate, evaporated, triturated with MTBE, filtered, dried in the oven at 40° C. overnight. The product (24.4 g, 92.4%) was obtained as off-white crystals; m. p. 150-152° C. ¹H NMR (300 MHz, DMSO-D6, δ): 8.92 (s, 1H), 8.45 (d, 2H, J=8.5 Hz), 7.99 (d, 2H, J=8.5 Hz), 7.68 (m, 1H), 7.51 (m, 1H), 7.46 (d, 2H, J=8.5 Hz), 7.21 ( m, 2H), 5.61 (s, 2H), 4.31 (m, 2H, J=7.2 Hz), 3.32 (s, 2H), 130 (t, 3H, J=7.2 Hz).

### Example 3

Step 1: Preparation of ethyl 2-amino-1,3-oxazole-4-carboxylate

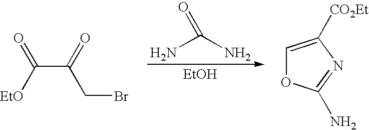

A stirred solution of ethyl bromopyruvate (50.2 g, 257.2 mmol) in EtOH is treated with urea (23.2 g, 385.8 mmol) refluxed overnight and concentrated under reduced pressure. The resultant residue is dissolved in EtOAC and water. The layers are separated and the aqueous layer washed with EtOAC. The combined organic layers are washed successively with saturated sodium chloride, dried over Mg₂SO₄ and concentrated under reduced pressure to give the title product.

Step 2: Preparation of ethyl 2-chloro-1,3-oxazole-4-carboxylate

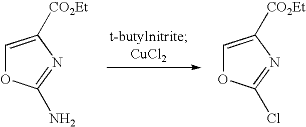

A stirred solution of tert-butyl nitrite (1.0 g, 9.61 mmol) and copper (II) chloride (1.29 g, 9.61 mmol) in CH₃CN is treated with ethyl 2-amino-1,3-oxazole-4-carboxylate (1.0 g, 6.4 mmol) stirred 2 hours at room temperature, heated to 80° C. for 30 minutes and concentrated under reduced pressure. The resultant residue is dissolved in EtOAc and water. The layers are separated and the organic layer washed with saturated sodium chloride, dried over Mg₂SO₄ and concentrated under reduced pressure to give the title product.

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A process for the preparation of a compound of formula I: 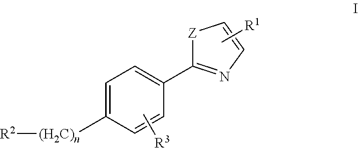
wherein,
R¹ is independently at each occurrence in the process H, halo, hydroxy, —CO₂(C₁-C₃ alkyl), C₁-C₆ alkyl, C₁-C₆ acyl, C₁-C₆ alkoxy, wherein each C₁-C₆ alkyl, C₁-C₆ acyl, C₁-C₆ alkoxy, is substituted with 0-4 substituents independently selected from the group consisting of C₁-C₄ alkyl, C₃-C₁₀ cycloakyl, C₂-C₆ alkenyl, C₂-C₆ alkynyl, halo, nitro, cyano, hydroxy, phenyl, 5-14 membered heterocyclyl or heteroaryl, —N(R^{a})₂, —C(O)R^{b}, —OR^{c} and —S(O)ₚR^{d};
R² is independently at each occurrence in the process a 5-14 membered heteroaryl substituted with 0-4 substituents independently selected from the group consisting of C₁-C₄ alkyl, C₃-C₁₀ cycloakyl, C₂-C₆ alkenyl, C₂-C₆ alkynyl, halo, nitro, cyano, hydroxy, phenyl, a 5-14 membered heterocyclyl or heteroaryl, —N(R^{a})₂, —C(O)R^{b}, —OR^{c} and —S(O)ₚR^{d};
R³ is independently at each occurrence in the process H, halo, nitro, cyano, hydroxy, S(O)ₚR^{d}, —N(R^{a})₂, C₁-C₆ alkyl, C₁-C₆ acyl, C₁-C₆ alkoxy, C₆-C₁₀ aryl, a 5-14 membered heteroaryl or heterocyclyl, or C₃-C₁₀ cycloalkyl, wherein each C₁-C₆ alkyl, C₁-C₆ acyl, C₁-C₆ alkoxy, C₆-C₁₀ aryl, 5-14 membered heteroaryl or heterocyclyl, or C₃-C₁₀ cycloalkyl is substituted with 0-4 substituents independently selected from the group consisting of C₁-C₄ alkyl, C₃-C₁₀ cycloakyl, C₂-C₆ alkenyl, C₂-C₆ alkynyl, halo, nitro, cyano, hydroxy, phenyl, a 5-14 membered heterocyclyl or heteroaryl, —N(R^{a})₂, —C(O)R^{b}, —OR^{c} and —S(O)ₚR^{d};
Z is O or S;
each R^{a} is independently H, C₁-C₄ alkyl optionally substituted with halo, phenyl, —CHO, —C(O)(C₁-C₄ alkyl) or —CO₂(C₁-C₄ alkyl);
each R^{b} is independently H, —OH, —O(C₁-C₄), C₁-C₄ alkyl optionally substituted with halo, phenyl, —NH₂, —NH(C₁-C₄ alkyl) or —N(C₁-C₄ alkyl)₂;
each R^{c} is independently H, C₁-C₄ alkyl, C₁-C₄ haloalkyl, phenyl, —CHO or —C(O)(C₁-C₄ alkyl);
each R^{d} is independently C₁-C₄ alkyl, C₁-C₄ haloalkyl, phenyl or —OH;
each p is independently 0, 1 or 2; and
n is 0 or 1; or
a tautomer, stereoisomer or pharmaceutically acceptable salt thereof;
wherein the process comprises reacting a compound of formula IB or tautomer thereof: 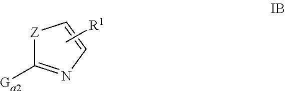
wherein Gₐ₂ is an activating group;
with a compound of formula IA: 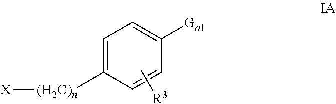
wherein,
Gₐ₁ is an activating group; and
(a) X is R², to form the compound of formula I; or
(b) X is Gₐ₃, thereby forming the compound of IC or tautomer thereof; or X is a hydroxy group and the process further comprises activating the hydroxy group X to form a compound of formula IC or tautomer thereof: 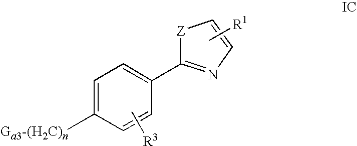
wherein,
Gₐ₃ is an activating group;
(i) optionally activating a compound of the formula H—R² to form activated-R²; and
(ii) reacting H—R² or activated-R² with the compound of formula IC to form the compound of formula I.

2. The process of claim 1, wherein R¹ is —CO₂(C₁-C₃ alkyl).

3. The process of claim 2, wherein the process further comprises:
reducing the —CO₂(C₁-C₃ alkyl) group in the compound of formula IC to form a reduced-R¹ group.

4. The process of claim 3, wherein the reducing step comprises contacting the —CO₂(C₁-C₃ alkyl) group with a reducing metal hydride; and
wherein the reduced-R¹ group is —CH₂OH.

5. The process of claim 4, wherein the reducing metal hydride is lithium aluminum hydride (LiAIH₄).

6. The process of claim 2, wherein the process further comprises:
reacting the —CO₂(C₁-C₃ alkyl) group in the compound of formula IC with a base to convert R¹ to a carboxylic acid.

7. The process of claim 2, wherein R¹ is —CO₂CH₂CH₃.

8. The process of claim 1, wherein Z is O.

9. The process of claim 1, wherein R² is benzimidazolyl substituted with 0-4 substituents independently selected from the group consisting of C₁-C₄ alkyl, C₃-C₁₀ cycloakyl, C₂-C₆ alkenyl, C₂-C₆ alkynyl, halo, nitro, cyano, hydroxy, phenyl, a 5-14 membered heterocyclyl or heteroaryl, —N(R^{a})₂, —C(O)R^{b}, —OR^{c} and —S(O)ₚR^{d}.

10. The process of claim 9, wherein R² is unsubstituted benzimidazol-1-yl.

11. The process of claim 1, wherein R³ is H.

12. The process of claim 1, wherein n is 1.

13. The process of claim 1, wherein each activating group is independently selected from the group consisting of halo, —B(OH)₂, tosylate, mesylate, and triflate.

14. The process of claim 1, wherein Gₐ₁ is —B(OH)₂.

15. The process of claim 1, wherein Gₐ₂ is chloro or bromo.

16. The process of claim 1, wherein the step of reacting a compound of formula IA with a compound of formula IB comprises a Suzuki coupling in the presence of a palladium catalyst.

17. The process of claim 16, wherein the palladium catalyst is not tetrakis(triphenylphosphine)palladium (0).

18. The process of claim 16, wherein the Suzuki coupling is performed in the presence of tris(dibenzylideneacetone) dipalladium (0) and tri(tertbutylphosphonium)tetrafluoroborate.

19. The process of claim 16, wherein the Suzuki coupling is performed in a solvent comprising aqueous dioxane and postassium carbonate.

20. The process of claim 1, wherein X is a hydroxy group.

21. The process of claim 20, wherein the step of activating the hydroxy group comprises contacting the hydroxyl group with a halogenating agent.

22. The process of claim 21, wherein the halogenating agent is thionyl chloride.

23. The process of claim 1, wherein step (b)(ii) comprises reacting H—R² with the compound of formula IC in the presence of a base.

24. The process of claim 23, wherein H—R² is 1H-benzo[d]imidazole and the base is sodium hydride (NaH).

25. The process of claim 1, wherein Gₐ₃ is chloro or bromo.

26. The process of claim 1, wherein, in the compound of formula IA, X is R²; and the process further comprises preparing a compound of formula IA by reacting H—R² or activated-R² with a compound of formula ID: 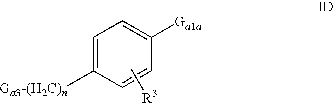
wherein,
Gₐ₃ is an activating group; and
Gₐ₁ₐ is the same activating group as Gₐ₁ in the compound of formula IA, thereby forming the compound of formula IA; or
Gₐ₁ₐ is a different activating group from Gₐ₁ in the compound of formula IA and the process further comprises converting Gₐ₁ₐ to Gₐ₁, thereby forming the compound of formula IA.

27. The process of claim 26, wherein Gₐ₁ₐ is bromo and Gₐ₁ in the compound of formula IA is —B(OH)₂.

28. The process of claim 26, wherein the converting step comprises reacting the compound of formula ID with a in the presence of tert-butyllithium and triisopropyl boronic acid (B(OiPr)₃).

29. The process of claim 26, wherein Gₐ₃ is bromo.

30. The process of claim 1, wherein R¹ is bound alpha to the N-position.

31. The process of claim 1, wherein any of the process steps is performed in a solvent which is independently a protic solvent, an aprotic solvent, a polar solvent, a nonpolar solvent, a protic polar solvent, an aprotic nonpolar solvent, or an aprotic polar solvent.

32. The process of claim 1, wherein any of the process steps includes a purification step comprising at least one of: filtration, extraction, chromatography, trituration, or recrystalization.

33. The process of claim 1, wherein any of the process steps comprises an analytical step comprising liquid chromatography (LC), mass spectroscopy (MS), liquid chromatography/mass spectroscopy (LC/MS), gas chromatography (GC), gas chromatography/mass spectroscopy (GC/MS), nuclear magnetic resonance (NMR), thin layer chromatography (TLC), melting point (MP) analysis, optical rotation (OR) or elemental analysis.
